(19) United States
(12) Patent Application Publication
(10) Pub. No.: US 2024/0339223 A1
(43) Pub. Date: Oct. 10, 2024

(54) RISK CALCULATION APPARATUS

(71) Applicant: Murata Manufacturing Co., Ltd., Kyoto (JP)

(72) Inventors: Hirofumi TSUCHIMOTO, Kyoto (JP); Kenta SUZUKI, Kyoto (JP); Masahiro SASAKI, Kyoto (JP); Masashi FURUKUBO, Kyoto (JP); Tatsuya OSAKI, Kyoto (JP)

(21) Appl. No.: 18/748,363

(22) Filed: Jun. 20, 2024

Related U.S. Application Data
(63) Continuation of application No. PCT/JP2022/043357, filed on Nov. 24, 2022.

(30) Foreign Application Priority Data
Dec. 28, 2021 (JP) 2021-215021

(51) Int. Cl. G16H 50/30; A61B 5/01

(52) U.S. Cl. CPC G16H 50/30; A61B 5/01

(57) ABSTRACT

A processor obtains a body temperature and patient condition information of a patient via a communication device. The processor calculates a comparison result by comparing the body temperature with a reference temperature obtained by the communication device and outputs a risk related to the health status of the patient based on the comparison result. The processor performs at least one of a process of correcting the body temperature based on the patient condition information when comparing the body temperature with the reference temperature, a process of setting the reference temperature based on the patient condition information when comparing the body temperature with the reference temperature, and a process of increasing or decreasing the comparison result based on the patient condition information.

### CROSS REFERENCE TO RELATED APPLICATION

This is a continuation of International Application No. PCT/JP2022/043357 filed on Nov. 24, 2022, which claims priority from Japanese Patent Application No. 2021-215021 filed on Dec. 28, 2021. The contents of these applications are incorporated herein by reference in their entireties.

### BACKGROUND OF THE DISCLOSURE

### Field of the Disclosure

The present disclosure relates to a risk calculation apparatus, a risk calculation system, and a program for calculating a risk related to the health status of a patient.

### Description of the Related Art

It is known to calculate a risk related to the health status of a patient based on a measured value of a parameter indicating a condition of the patient to detect the onset or the worsening of a disease of the patient at an early stage.

For example, Patent Document 1 discloses a patient monitoring system that determines a patient status value based on measured values of one or more physiological parameters.

Patent Document 1: Japanese Unexamined Patent Application Publication (Translation of PCT Application) No. 2018-506759

### BRIEF SUMMARY OF THE DISCLOSURE

A parameter measured to calculate a risk related to the health status of a patient may vary due to factors that are not directly related to the disease of the patient. This may lead to a problem in which the risk related to the health status of the patient is calculated incorrectly. Furthermore, even when the measured value of a parameter is the same, the magnitude of the risk varies depending on the type of disease that a patient has or is suspected to have. Therefore, it is required to accurately calculate a risk related to the health status of a patient regardless of the condition of the patient that depends on factors related to and unrelated to a disease of the patient.

One possible benefit of the present disclosure is to provide a risk calculation apparatus that can calculate a risk related to the health status of a patient more accurately than the related art regardless of the condition of the patient.

An aspect of the present disclosure provides a risk calculation apparatus that calculates a risk related to the health status of a patient. The risk calculation apparatus includes an input unit that obtains a body temperature and patient condition information of the patient, and a calculation unit that calculates a comparison result by comparing the body temperature obtained by the input unit with a reference temperature and outputs the risk related to the health status of the patient based on the comparison result. The calculation unit performs at least one of a process of correcting the body temperature based on the patient condition information when comparing the body temperature with the reference temperature, a process of setting the reference temperature based on the patient condition information when comparing the body temperature with the reference temperature, and a process of increasing or decreasing the comparison result based on the patient condition information.

According to an aspect of the present disclosure, the patient condition information includes variation according to a circadian rhythm in the body temperature of the patient; and the calculation unit performs, based on the variation according to the circadian rhythm, at least one of the process of correcting the body temperature, the process of setting the reference temperature, and the process of increasing or decreasing the comparison result.

According to an aspect of the present disclosure, the calculation unit performs at least one of the process of correcting the body temperature, the process of setting the reference temperature, and the process of increasing or decreasing the comparison result to offset the variation according to the circadian rhythm.

According to an aspect of the present disclosure, the calculation unit performs at least one of the process of correcting the body temperature, the process of setting the reference temperature, and the process of increasing or decreasing the comparison result to follow the variation according to the circadian rhythm.

According to an aspect of the present disclosure, the input unit continuously obtains the patient condition information; and the calculation unit performs, based on a time when the input unit obtains the body temperature of the patient, at least one of the process of correcting the body temperature, the process of setting the reference temperature, and the process of increasing or decreasing the comparison result.

According to an aspect of the present disclosure, the patient condition information includes activity information indicating whether the patient is currently asleep or awake; and the calculation unit performs, based on the activity information, at least one of the process of correcting the body temperature, the process of setting the reference temperature, and the process of increasing or decreasing the comparison result.

According to an aspect of the present disclosure, the patient condition information includes a basal metabolic rate of the patient or information for calculating the basal metabolic rate of the patient; and the calculation unit performs, based on the basal metabolic rate of the patient and a standard basal metabolism, at least one of the process of correcting the body temperature, the process of setting the reference temperature, and the process of increasing or decreasing the comparison result.

According to an aspect of the present disclosure, the calculation unit performs at least one of the process of correcting the body temperature, the process of setting the reference temperature, and the process of increasing or decreasing the comparison result to offset an increase or decrease in the basal metabolic rate of the patient from the standard basal metabolism.

According to an aspect of the present disclosure, the calculation unit performs at least one of the process of correcting the body temperature, the process of setting the reference temperature, and the process of increasing or decreasing the comparison result to follow an increase or decrease in the basal metabolic rate of the patient from the standard basal metabolism.

According to an aspect of the present disclosure, the patient condition information includes medication information indicating a drug administered to the patient; and the calculation unit performs, based on the medication information, at least one of the process of correcting the body temperature, the process of setting the reference temperature, and the process of increasing or decreasing the comparison result.

According to an aspect of the present disclosure, the calculation unit performs at least one of the process of correcting the body temperature, the process of setting the reference temperature, and the process of increasing or decreasing the comparison result to offset an increase or decrease in the body temperature of the patient caused by the drug.

According to an aspect of the present disclosure, the calculation unit performs at least one of the process of correcting the body temperature, the process of setting the reference temperature, and the process of increasing or decreasing the comparison result to follow an increase or decrease in the body temperature of the patient caused by the drug.

According to an aspect of the present disclosure, the medication information includes at least one of a type of the drug, elapsed time after the drug is administered, and an administration method of the drug.

According to an aspect of the present disclosure, the patient condition information includes a result of diagnosis of a disease of the patient; and the calculation unit performs, based on the result of diagnosis, at least one of the process of correcting the body temperature, the process of setting the reference temperature, and the process of increasing or decreasing the comparison result.

According to an aspect of the present disclosure, the patient condition information includes a pulse of the patient; and the calculation unit calculates relative bradycardia based on the pulse and performs, based on the relative bradycardia, at least one of the process of correcting the body temperature, the process of setting the reference temperature, and the process of increasing or decreasing the comparison result.

According to an aspect of the present disclosure, the calculation unit performs at least one of the process of correcting the body temperature, the process of setting the reference temperature, and the process of increasing or decreasing the comparison result to follow a difference between a first body temperature increase actually measured and a second body temperature increase estimated based on the pulse.

According to an aspect of the present disclosure, the calculation unit performs at least one of the process of correcting the body temperature, the process of setting the reference temperature, and the process of increasing or decreasing the comparison result to offset a difference between a first body temperature increase actually measured and a second body temperature increase estimated based on the pulse.

According to an aspect of the present disclosure, a risk calculation system includes a temperature sensor that measures the body temperature of the patient, and the risk calculation apparatus.

An aspect of the present disclosure provides a program including instructions that are executable by a processor of a computer to calculate a risk related to the health status of a patient. The instructions cause the processor to perform a first step of obtaining a body temperature and patient condition information of the patient, and a second step of comparing the body temperature of the patient with a reference temperature to calculate a comparison result and calculating the risk related to the health status of the patient based on the comparison result. The second step includes at least one of a process of correcting the body temperature based on the patient condition information when comparing the body temperature with the reference temperature, a process of setting the reference temperature based on the patient condition information when comparing the body temperature with the reference temperature, and a process of increasing or decreasing the comparison result based on the patient condition information.

An aspect of the present disclosure makes it possible to calculate a risk related to the health status of a patient more accurately than the related art regardless of the condition of the patient.

### BRIEF DESCRIPTION OF THE SEVERAL VIEWS OF THE DRAWINGS

FIG. 1 is a block diagram illustrating a configuration of a risk calculation system according to a first embodiment.

FIG. 2 is a flowchart illustrating a first example of a risk calculation process that is performed by a processor 11 illustrated in FIG. 1 and includes a body temperature correction process.

FIG. 3 is a flowchart illustrating, as a first example of step S102 in FIG. 2, a subroutine corresponding to a body temperature correction process based on a circadian rhythm.

FIG. 4 is a drawing used to describe the fitting of a circadian rhythm to body temperatures measured over a 24-hour period.

FIG. 5 is a drawing used to describe the body temperature correction process based on a circadian rhythm illustrated in FIG. 3.

FIG. 6 is a flowchart illustrating, as a second example of step S102 in FIG. 2, a subroutine corresponding to a body temperature correction process based on a basal metabolic rate.

FIG. 7 is a flowchart illustrating, as a third example of step S102 in FIG. 2, a subroutine corresponding to a body temperature correction process based on medication.

FIG. 8 is a drawing showing the concentration of a drug in the blood in relation to the elapsed time after a drug is administered to a patient.

FIG. 9 is a drawing used to describe the body temperature correction process based on medication illustrated in FIG. 7.

FIG. 10 is a flowchart illustrating, as a fourth example of step S102 in FIG. 2, a subroutine corresponding to a body temperature correction process based on a result of diagnosis.

FIG. 11 is a block diagram illustrating a configuration of a risk calculation system according to a variation of the first embodiment.

FIG. 12 is a flowchart illustrating, as a fifth example of step S102 in FIG. 2, a subroutine corresponding to a body temperature correction process based on a pulse.

FIG. 13 is a graph used to describe the occurrence of relative bradycardia.

FIG. 14 is a flowchart illustrating a second example of a risk calculation process that is performed by the processor 11 illustrated in FIG. 1 and includes a set value correction process.

FIG. 15 is a flowchart illustrating, as a first example of step S202 in FIG. 14, a subroutine corresponding to a set value correction process based on a circadian rhythm.

FIG. 16 is a flowchart illustrating, as a second example of step S202 in FIG. 14, a subroutine corresponding to a set value correction process based on a basal metabolic rate.

FIG. 17 is a flowchart illustrating, as a third example of step S202 in FIG. 14, a subroutine corresponding to a set value correction process based on medication.

FIG. 18 is a flowchart illustrating, as a fourth example of step S202 in FIG. 14, a subroutine corresponding to a set value correction process based on a result of diagnosis.

FIG. 19 is a flowchart illustrating, as a fifth example of step S202 in FIG. 14, a subroutine corresponding to a set value correction process based on a pulse.

FIG. 20 is a flowchart illustrating a third example of a risk calculation process that is performed by the processor 11 illustrated in FIG. 1 and includes a risk value correction process.

FIG. 21 is a flowchart illustrating, as a first example of step S303 in FIG. 20, a subroutine corresponding to a risk value correction process based on a circadian rhythm.

FIG. 22 is a flowchart illustrating, as a second example of step S303 in FIG. 20, a subroutine corresponding to a risk value correction process based on a basal metabolic rate.

FIG. 23 is a flowchart illustrating, as a third example of step S303 in FIG. 20, a subroutine corresponding to a risk value correction process based on medication.

FIG. 24 is a flowchart illustrating, as a fourth example of step S303 in FIG. 20, a subroutine corresponding to a risk value correction process based on a result of diagnosis.

FIG. 25 is a flowchart illustrating, as a fifth example of step S303 in FIG. 20, a subroutine corresponding to a risk value correction process based on a pulse.

FIG. 26 is a block diagram illustrating a configuration of a risk calculation system according to a second embodiment.

### DETAILED DESCRIPTION OF THE DISCLOSURE

Risk calculation systems according to embodiments of the present disclosure are described below with reference to the drawings. Throughout the drawings, the same reference number indicates the same component.

### First Embodiment

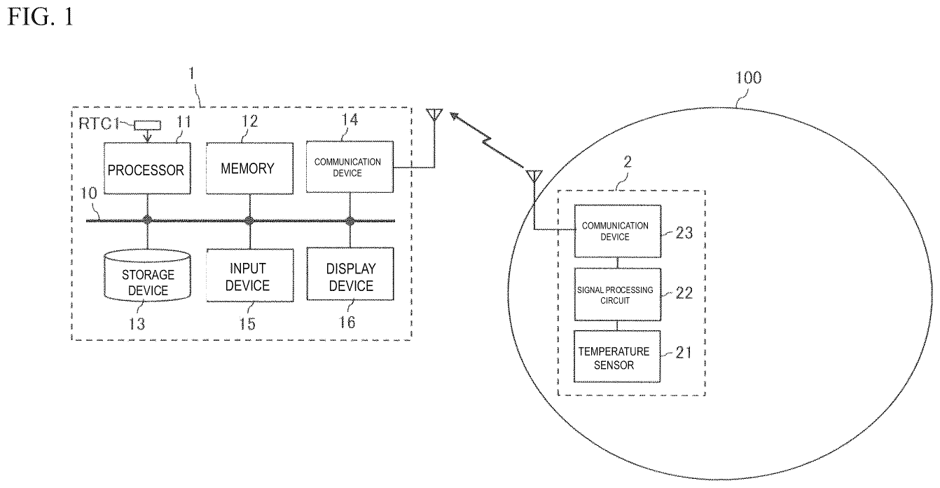

FIG. 1 is a block diagram illustrating a configuration of a risk calculation system according to a first embodiment. A risk calculation apparatus 1 obtains the body temperature of a patient 100 from a thermometer 2 attached to the body of the patient 100 and calculates risks related to the health status of the patient 100 based on the body temperature of the patient 100. Here, the “risks” include a prediction of the possibility that the patient 100 becomes seriously ill after a predetermined period of time (for example, half a day later).

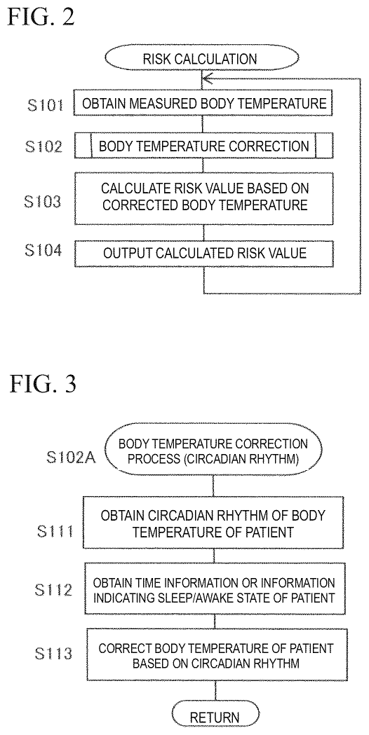

The risk calculation apparatus 1 includes a bus 10, a processor 11, a memory 12, a storage device 13, a communication device 14, an input device 15, a display device 16, and a clock RTC1. The processor 11 performs a risk calculation process, which is described later with reference to FIG. 2 and so on, to calculate a risk related to the health status of the patient 100 and also controls the overall operation of the risk calculation apparatus 1. The memory 12 temporarily stores programs and data necessary for the operation of the risk calculation apparatus 1. The storage device 13 is a non-volatile storage medium that stores programs and data necessary for the operation of the risk calculation apparatus 1. The communication device 14 is connected for communication to a thermometer 2 and obtains the body temperature of the patient 100 from the thermometer 2. The input device 15 receives user inputs for controlling the operations of the risk calculation apparatus 1. The input device 15 includes, for example, a keyboard and a pointing device. The display device 16 displays the calculated risk related to the health status of the patient 100. The clock RTC1 provides time information indicating the current time. The processor 11, the memory 12, the storage device 13, the communication device 14, the input device 15, and the display device 16 are connected to each other via the bus 10.

The risk calculation apparatus 1 may be a tablet, notebook, or desktop general-purpose personal computer or may be a dedicated computing device, such as a wearable computing device. Also, the risk calculation apparatus 1 may be an integrated apparatus including a combination of multiple components. For example, the risk calculation apparatus 1 may be a desktop computer including a main unit, a display (display device), and a keyboard (input device).

The thermometer 2 includes a temperature sensor 21, a signal processing circuit 22, and a communication device 23. The temperature sensor 21 detects the body temperature of the patient 100. The signal processing circuit 22 converts the body temperature of the patient 100 detected by the temperature sensor 21 into a format (for example, a digital value) that can be transmitted to the risk calculation apparatus 1. The communication device 23 is connected for communication to the risk calculation apparatus 1 and transmits the body temperature of the patient 100 to the risk calculation apparatus 1.

The thermometer 2 may also be a core body thermometer that measures the core body temperature of the patient 100 by detecting the temperature of, for example, the trunk, the eardrum, the rectum, or the esophagus. The thermometer 2 may be wirelessly connected to the risk calculation apparatus 1 via, for example, Bluetooth (registered trademark) or WiFi (registered trademark), or may be connected by wire to the risk calculation apparatus 1.

The processor 11 obtains the current body temperature of the patient 100 from the thermometer 2 via the communication device 14.

The processor 11 obtains patient condition information regarding the condition of the patient 100 other than the current body temperature via the input device 15 and stores the obtained patient condition information in the storage device 13. Also, the processor 11 may obtain, via the communication device 14, patient condition information stored in advance in an external server apparatus (not shown) that is connected for communication to the risk calculation apparatus 1 via the communication device 14. The patient condition information includes, for example, at least one of the circadian rhythm of the body temperature of the patient 100, the basal metabolic rate or information associated with the basal metabolic rate of the patient 100, medication information regarding drugs administered to the patient 100, a result of diagnosis of a disease of the patient 100, and the pulse of the patient 100. The patient condition information also includes the current body temperature of the patient 100 obtained from the thermometer 2 as described above.

The processor 11 calculates a comparison result by comparing the current body temperature of the patient 100 with a set value, and determines and outputs a risk related to the health status of the patient 100 based on the comparison result.

The set value is a reference temperature. For example, the set value may be set to a temperature threshold, such as 38.5° C., that is significantly higher than the normal body temperature of the patient 100, or may be set to the normal body temperature of the patient 100, such as 36.5° C. Also, the set value is not necessarily one reference temperature but may be defined by a pair of reference temperatures that indicate the upper and lower limits of a certain temperature range. The set value may also represent multiple pairs of reference temperatures indicating multiple temperature ranges that are different from each other.

The comparison result between the current body temperature of the patient 100 and the set value may be expressed, for example, in the form of a numerical value. In the present application, a numerical value indicating the comparison result is hereafter referred to as a “risk value”. The risk value may be represented by a discrete value or a continuous value. For example, the set value may be set to a temperature threshold significantly higher than the normal body temperature of the patient 100. In this case, when the body temperature of the patient 100 exceeds the temperature threshold, it may be determined that the patient 100 is in a high risk state, and the risk value may be set to 1; otherwise, the risk value may be set to 0. Also, multiple temperature thresholds may be set such that the risk value increases as the body temperature increases. Also, when the set value is set to the normal body temperature of the patient 100, the risk value may be calculated to indicate the difference between the current body temperature and the normal body temperature of the patient 100. In this case, the risk value may also be calculated to increase as the difference, by which the current body temperature of the patient 100 exceeds the normal body temperature, increases.

Based on the patient condition information, the processor 11 performs at least one of correcting the current body temperature, setting or resetting the set value, and increasing or decreasing the risk value (or the comparison result). In the present application, these changes (correction, setting, and increasing/decreasing) to the parameters are collectively referred to as “correcting the risk”. In other words, in the present embodiment, “correcting the risk” not only indicates directly correcting the risk related to the health status of the patient 100, which is information to be ultimately presented to the user, but also indicates indirectly correcting the risk, i.e., correcting a parameter used to calculate the risk. “Correcting the risk” includes correcting the body temperature and calculating a risk value based on the corrected body temperature, setting or resetting a set value and calculating a risk value using the set value that is set or reset, and increasing or decreasing a calculated risk value.

Based on the calculated risk value, the processor 11 determines and outputs a risk related to the health status of the patient 100. The processor 11 outputs the determined risk to the display device 16. The processor 11 may also output the determined risk to an external device that is connected via the communication device 14 to the risk calculation apparatus 1 for communication. The processor 11 may also output the determined risk via a speaker (not shown).

The processor 11 may output the calculated risk value itself as the risk related to the health status of the patient 100 or may output any other numerical value (such as a percentage) obtained by converting the risk value. Furthermore, the processor 11 may output the determined risk in a form, such as a visual or auditory form, other than a numerical form. For example, the processor 11 may output the determined risk in text, such as “high”, “intermediate”, or “low”. Also, the processor 11 may display the determined risk with gradations of color that change as the measured body temperature deviates from a reference value, such as the normal body temperature. Furthermore, when the determined risk indicates that the patient 100 is in a high risk state, the processor 11 may output an alarm via a speaker (not shown).

In the descriptions below, for brevity, it is assumed that the processor 11 outputs the calculated risk value itself as the risk related to the health status of the patient 100. However, the processor 11 may output the determined risk in any other form as described above.

The communication device 14 is an example of an input unit that obtains the current body temperature of the patient 100. The input device 15 or the communication device 14 is an example of an input unit that obtains the patient condition information. The processor 11 is an example of a calculation unit that determines a risk related to the health status of the patient 100. The display device 16, the communication device 14, or the speaker (not shown) is an example of an output unit that outputs a determined risk.

As described above, the processor 11 corrects the current body temperature, the set value, or the risk value based on the patient condition information. Below, a risk calculation process including a body temperature correction process, a risk calculation process including a set value correction process, and a risk calculation process including a risk value correction process are described.

### Risk Calculation Process Including Body Temperature Correction Process

FIG. 2 is a flowchart illustrating a first example of a risk calculation process that is performed by the processor 11 illustrated in FIG. 1 and includes a body temperature correction process. According to the process of FIG. 2, the processor 11 corrects a measured current body temperature of the patient 100 before calculating a risk value.

At step S101, the processor 11 obtains a measured current body temperature of the patient 100 from the thermometer 2 via the communication device 14.

At step S102, the processor 11 performs the body temperature correction process to correct the current body temperature of the patient 100 based on patient condition information.

At step S103, the processor 11 calculates a risk value based on the corrected body temperature and a set value.

At step S104, the processor 11 outputs the calculated risk value to the display device 16.

The processor 11 periodically repeats the risk calculation process of FIG. 2.

As described above, the patient condition information includes, for example, at least one of a circadian rhythm of the body temperature of the patient 100, a basal metabolic rate of the patient 100 or information associated the basal metabolic rate, medication information about a drug administered to the patient 100, a result of diagnosis of a disease of the patient 100, and a pulse of the patient 100. Below, a body temperature correction process based on the circadian rhythm, a body temperature correction process based on the basal metabolic rate, a body temperature correction process based on the medication, a body temperature correction process based on the result of diagnosis, and a body temperature correction process based on the pulse are described.

### Body Temperature Correction Process Based on Circadian Rhythm

The circadian rhythm is circadian variation of a parameter indicating a condition of a human that exists independently of a disease and the degree of the disease. The body temperature of a patient may increase or decrease due to the circadian rhythm independently of a disease and the degree of the disease, and therefore, the risk related to the health status of the patient may be miscalculated. Below, a method of correcting the body temperature of a patient to reduce the influence of the circadian rhythm is described.

FIG. 3 is a flowchart illustrating, as a first example of step S102 in FIG. 2, a subroutine corresponding to the body temperature correction process based on the circadian rhythm. To distinguish the body temperature correction process based on the circadian rhythm from body temperature correction processes based on other types of patient condition information, the step of the body temperature correction process of FIG. 3 is represented by a reference number S102A.

At step S111, the processor 11 reads the circadian rhythm of the body temperature of the patient 100 from the storage device 13. The circadian rhythm of the body temperature of the patient 100 may be generated and stored in the storage device 13 in advance by the processor 11 based on a log of the body temperature of the patient 100 obtained by using the thermometer 2 over a time period greater than or equal to 24 hours. Also, the circadian rhythm of the body temperature of the patient 100 may be obtained in advance from an external server apparatus (not shown) via the communication device 14 and stored in the storage device 13.

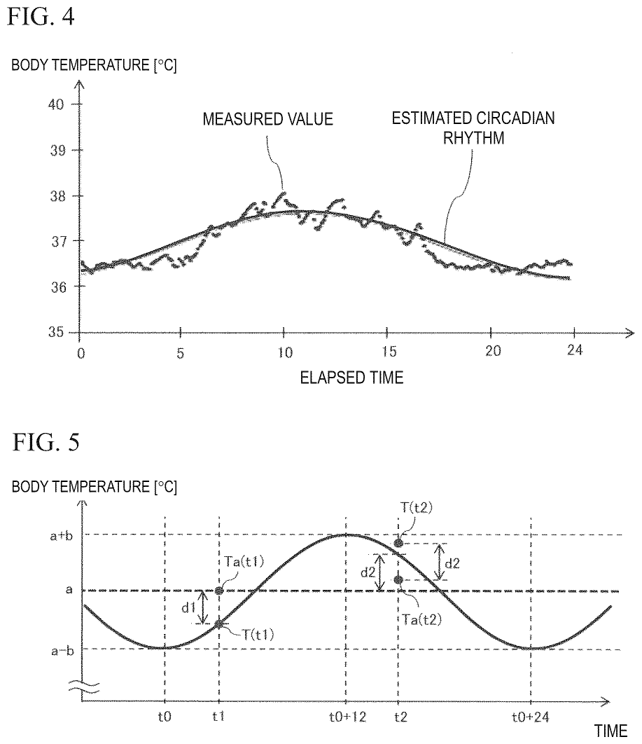

FIG. 4 is a drawing used to describe the fitting of a circadian rhythm to body temperatures measured over a 24-hour period. The human body temperature generally reaches the maximum value while awake and reaches the minimum value while asleep. Also, the time when the body temperature reaches the maximum value and the time when the body temperature reaches the minimum value depend on the wake-up time and the bedtime of a subject. However, in general, the wake-up time and the bedtime of the subject are unknown, and the average value, the amplitude, and the initial phase of the body temperature are also unknown. According to the example in FIG. 4, a circadian rhythm function x(t) of the body temperature of the subject is estimated by a formula below by turning the measured body temperature (actual value) into a cosine wave (or a sine wave).

$$x ( t ) = a - b \times cos ( 2 Π ( t - t0 ) / 24 )$$

In the formula, “a” indicates the average value of the body temperature, “b” indicates the amplitude of change in the body temperature, and “t0” indicates the initial phase, that is, the time (unit:hour) at which the body temperature reaches the minimum value.

The circadian rhythm may be expressed not only by a cosine wave (or a sine wave) but also by, for example, a triangular wave or a rectangular wave. Furthermore, the circadian rhythm is not necessarily generated based on a log of the body temperature of the patient 100 but may also be determined based on, for example, the wake-up time and the bedtime of the patient 100 obtained through an interview with a doctor.

At step S112, the processor 11 obtains time information indicating the current time from the clock RTC1. When the circadian rhythm of the body temperature of the patient 100 is represented by a rectangular wave that changes to a low level while asleep and to a high level while awake, the processor 11 may obtain, instead of the time information, activity information indicating whether the patient 100 is currently asleep or awake via the input device 15.

At step S113, the processor 11 corrects the current body temperature of the patient 100 based on the circadian rhythm and the current time (in other words, the time when the body temperature of the patient 100 was acquired) to offset the variation in the body temperature of the patient 100 according to the circadian rhythm. A corrected body temperature Ta(t) is calculated by a formula below based on a current body temperature T(t) and the circadian rhythm function x(t) described above.

$$T a ( t ) = T ( t ) - ( x ( t ) - a ) = T ( t ) + b \times cos ( 2 Π ( t - t 0 ) / 24 )$$

FIG. 5 is a drawing used to describe the body temperature correction process based on the circadian rhythm illustrated in FIG. 3. The processor 11 generally corrects the measured body temperature T(t) to approach the average value “a” of the body temperature according to the circadian rhythm. At time t1, the body temperature according to the circadian rhythm is lower than the average value “a” by a temperature d1. Therefore, the processor 11 calculates a corrected body temperature Ta(t1) by adding the temperature d1 to a body temperature T(t1) measured at time t1. Also, at time t2, the body temperature according to the circadian rhythm is higher than the average value “a” by the temperature d1. Therefore, the processor 11 calculates a corrected body temperature Ta(t2) by subtracting the temperature d1 from a body temperature T(t2) measured at time t2.

Here, “offset the variation in the body temperature” indicates at least partially offsetting an increase or decrease in the body temperature according to the circadian rhythm. Referring to the example in FIG. 5, the body temperature T(t1) measured at time t1 is on the plot of the circadian rhythm, but the body temperature T(t2) measured at time t2 is off the plot of the circadian rhythm. In the former case, the corrected body temperature Ta(t1) matches the average value “a” of the body temperature according to the circadian rhythm. However, in the latter case, the corrected body temperature Ta (t2) does not match the average value “a” of the body temperature according to the circadian rhythm.

As described above, when the circadian rhythm of the body temperature of the patient 100 is represented by a rectangular wave, the processor 11, at step S113, may correct the current body temperature of the patient 100 based on the circadian rhythm and the activity information to offset the variation in the body temperature of the patient 100 according to the circadian rhythm.

Performing the body temperature correction process based on the circadian rhythm makes it possible to reduce the influence of the circadian variation in the body temperature of the patient 100 and thereby makes it possible to calculate a risk related to the health status of the patient 100 more accurately than the related art regardless of the time of day.

### Body Temperature Correction Process Based on Basal Metabolic Rate

While the body temperature of a person has a high correlation with the basal metabolic rate of the person, the basal metabolic rate of a person varies depending on the body weight, body height, age, and gender of the person. These information items can be used as information for calculating the basal metabolism. In other words, the body temperature of a patient may increase or decrease due to the individual variation of the basal metabolic rate and independent of a disease and the degree of the disease. This may lead to a problem in which the risk related to the health status of the patient is calculated incorrectly. Below, a method of correcting the body temperature of the patient to reduce the influence of the individual variation in the basal metabolic rate is described.

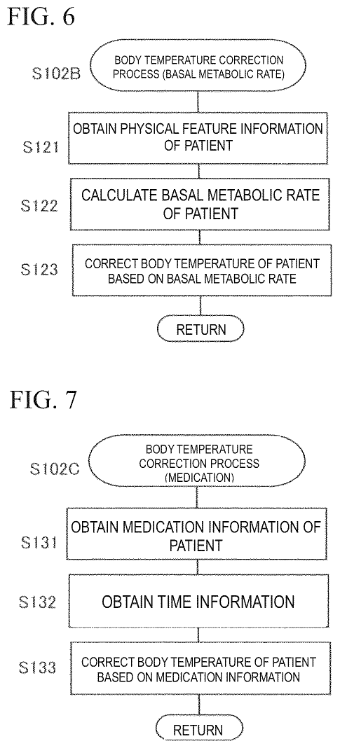

FIG. 6 is a flowchart illustrating, as a second example of step S102 in FIG. 2, a subroutine corresponding to a body temperature correction process based on a basal metabolic rate. To distinguish the body temperature correction process based on the basal metabolic rate from body temperature correction processes based on other types of patient condition information, the step of the body temperature correction process of FIG. 6 is represented by a reference number S102B.

At step S121, the processor 11 obtains physical feature information of the patient 100. The physical feature information of the patient 100 includes, for example, at least one of body weight, body height, age, and gender. The processor 11 may read pre-stored physical feature information from the storage device 13, may obtain physical feature information via the input device 15, or may obtain physical feature information from an external server apparatus (not shown) via the communication device 14.

At step S122, the processor 11 calculates the basal metabolic rate of the patient 100 based on the physical feature information of the patient 100. Below are examples of known formulas for calculating a basal metabolic rate. The formulas were obtained by National Institute of Health and Nutrition of Japan.

$$Jm = ( 0.4 \; 81 \times W + 0. 0234 \times H - 0.0138 \times A - 0.4235 ) \times 1000 / 4.186 \\ Jf = ( 0.481 \times W + 0. 0234 \times H - 0.0138 \times A - 0.9708 ) \times 1000 / 4.186$$

Here, Jm represents a basal metabolic rate of a male, Jf represents a basal metabolic rate of a female, W represents a body weight, H represents a body height, and A represents an age. According to the above formulas, each of the basal metabolic rates Jm and Jf increases as the body weight W and the body height H increase, and decreases as the age A increases.

At step S123, the processor 11 corrects the current body temperature of the patient 100 based on the basal metabolic rate to offset the increase or decrease in the basal metabolic rate of the patient 100 from the standard basal metabolic rate. A corrected body temperature Tb is calculated by one of the formulas below based on the current body temperature T and the basal metabolic rate Jm or Jf.

$$Tb = T \times Mm / Jm ( for male ) \\ Tb = T \times Mf / Jf ( for female )$$

Here, Mm indicates an average basal metabolic rate for males (in other words, a standard basal metabolic rate for males), and Mf indicates an average basal metabolic rate for females (in other words, a standard basal metabolic rate for females).

According to the above formulas, the corrected body temperature Tb decreases as the body weight W and the body height H increase, and increases as the age A increases.

Here, “offset the increase or decrease in the basal metabolic rate” indicates at least partially offsetting the increase or decrease in the body temperature due to the individual variation in the basal metabolic rate.

When any of the body weight, the body height, and the age is unknown, the processor 11 may use an average value in a given population.

The processor 11 may obtain the physical feature information for calculating the basal metabolic rate (i.e., information associated with the basal metabolic rate of the patient 100) as described above and may instead obtain a pre-calculated basal metabolic rate itself. In this case, the processor 11 may read a pre-stored basal metabolic rate from the storage device 13, may obtain a basal metabolic rate via the input device 15, or may obtain a basal metabolic rate from an external server apparatus (not shown) via the communication device 14.

Performing the body temperature correction process based on the basal metabolic rate makes it possible to reduce the influence of the individual variation in the basal metabolic rate and thereby makes it possible to calculate a risk related to the health status of the patient 100 more accurately than the related art.

### Body Temperature Correction Process Based on Medication

The body temperature of a patient can be artificially decreased or increased by administering a drug to the patient. For example, when an antipyretic is administered to a patient who has a fever due to an illness, such as an infectious disease, the fever is reduced and the body temperature decreases due to the effect of the antipyretic for a certain period of time after the administration. However, because the antipyretic does not cure the illness itself, the risk related to the health status of the patient may be incorrectly calculated based on the body temperature of the patient. Below, a method of correcting the body temperature of a patient to reduce the influence of a drug administered to the patient is described.

FIG. 7 is a flowchart illustrating, as a third example of step S102 in FIG. 2, a subroutine corresponding to a body temperature correction process based on medication. To distinguish the body temperature correction process based on medication from body temperature correction processes based on other types of patient condition information, the step of the body temperature correction process of FIG. 7 is represented by a reference number S102C.

At step S131, the processor 11 obtains medication information regarding a drug administered to the patient 100. The medication information includes at least one of the type of drug, the elapsed time after the administration of the drug, and the administration method of the drug. Depending on the type of drug, the effect of the drug on the body temperature varies (fall or rise), and the duration of the effect of the drug also varies. Also, depending on the administration method of the drug, the time necessary for the onset of the effect varies, and the duration of the effect of the drug also varies. Examples of administration methods include oral administration, injection, and suppository. In the case of oral administration, the time necessary for the onset of the effect and the duration of the effect vary depending also on the dosage form (tablet, granules, powder, etc.). The processor 11 may read pre-stored medication information from the storage device 13, may obtain medication information via the input device 15, or may obtain medication information from an external server apparatus (not shown) via the communication device 14. When obtaining medication information via the input device 15, the processor 11 may display, on the display device 16, a user interface, such as a pull-down menu including multiple options, that facilitates the input of the medication information.

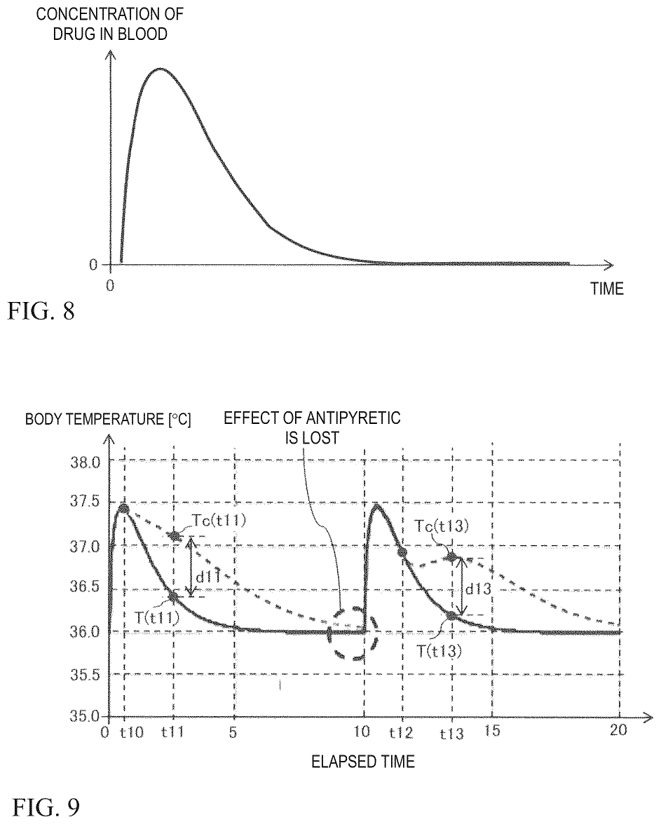

FIG. 8 is a drawing showing the concentration of a drug in the blood in relation to the elapsed time after the drug is administered to a patient. The concentration of the drug in the blood in relation to the elapsed time increases rapidly after the administration and decreases slowly after reaching the peak value. The concentration of the drug in the blood in relation to the elapsed time is represented by, for example, a Weibull distribution.

The amount of change in the body temperature due to the administration of a drug to the patient 100 depends on the concentration of the drug in the blood. Therefore, the profile of the body temperature change of the patient 100 has a shape similar to the shape of the profile of the concentration change in relation to the elapsed time as shown in FIG. 8.

Examples of antipyretics include ibuprofen, naproxen, ketoprofen, nimesulide, acetylsalicylic acid, and acetaminophen.

On the other hand, when a drug not for reducing fever is administered to the patient 100, the body temperature of the patient 100 may increase due to the side effect of the drug. Examples of drugs that increase the body temperature of the patient 100 are listed below.

(1) Drugs that cause high body temperature due to muscle hyperactivity: amphetamine, monoamine oxidase inhibitor, cocaine, lithium, antipsychotic (butyrophenone antipsychotic, phenothiazine antipsychotic), tricyclic or tetracyclic antidepressant, halothane, succinylcholine, MDMA, lysergic acid diethylamide (LSD), phenylcyclohexyl piperidine (PCP), strychnine, isoniazid, and sympathetic nerve activator (theophylline, ephedrine, etc.)

(2) Drugs that cause high body temperature due to hypermetabolism: salicylic acid, thyroid hormone, sympathetic nerve activator, alcohol withdrawal, sedative, and hypnotic withdrawal

(3) Drugs that cause high body temperature due to body temperature center disorder: alcohol, antipsychotic (phenothiazine antipsychotic), and inhaled or intravenous anesthetic

(4) Drugs that cause high body temperature due to heat dissipation disorder: anticholinergic drug, muscle relaxant, antipsychotic, sympathetic nerve activator

The medication information includes, for each drug, a profile of the body temperature change in relation to the elapsed time, i.e., the increase, the decrease, and the rate of increase or decrease in the body temperature.

At step S132, the processor 11 obtains time information indicating the current time from the clock RTC1.

At step S133, the processor 11 corrects the current body temperature of the patient 100 based on the medication information to offset the increase or decrease in the body temperature of the patient 100 caused by the drug. As described above, when the concentration of the drug in the blood in relation to the elapsed time is represented by a Weibull distribution, a corrected body temperature Tc(t) is calculated by the following formula based on the current body temperature T(t).

$$Tc ( t ) = T ( t ) \times ( 1 + κ \times ( α / {β}^{α} ) \times {T ( t )}^{α - 1} \times exp ( - {( T ( t ) / β )}^{α} ) )$$

Here, α indicates a shape coefficient, β indicates a scale coefficient, and κ indicates a correction coefficient.

Here, “offset the increase or decrease in the body temperature of the patient 100 caused by the drug” indicates at least partially offsetting the increase or decrease in the body temperature caused by the drug.

FIG. 9 is a drawing used to describe the body temperature correction process based on medication illustrated in FIG. 7. In FIG. 9, a thick solid line indicates the measured body temperature T(t), and a thick dashed line indicates the corrected body temperature Tc(t). At time 0, the body temperature of the patient 100 starts to increase. When an antipyretic is administered to the patient 100 at time t10, the body temperature gradually decreases. After ten hours from when the body temperature of the patient 100 starts to increase, the effect of the antipyretic is lost, and the body temperature of the patient 100 starts to increase again. Thereafter, when the antipyretic is administered to the patient 100 at time t12, the body temperature gradually decreases. The processor 11 calculates a corrected body temperature Tc(t11) by adding a temperature d11 to a body temperature T(t11) measured at time t11. The processor 11 calculates a corrected body temperature Tc(t13) by adding the temperature d13 to a body temperature T(t13) measured at time t13.

When an antipyretic is administered to the patient 100, the processor 11 corrects a measured body temperature by increasing the measured body temperature as shown in FIG. 9. In contrast, when a drug that increases the body temperature is administered to the patient 100, the processor 11 corrects a measured body temperature by decreasing the measured body temperature.

Performing the body temperature correction process based on medication makes it possible to reduce the influence of a drug administered to the patient 100 and thereby makes it possible to calculate a risk related to the health status of the patient 100 more accurately than the related art.

### Body Temperature Correction Process Based on Result of Diagnosis

Even when the measured body temperature is the same, the magnitude of the risk may vary depending on the type of disease that a patient has or is suspected to have. Below, a method of correcting the body temperature of the patient to reduce the influence of each type of disease is described.

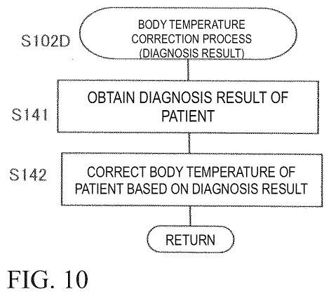

FIG. 10 is a flowchart illustrating, as a fourth example of step S102 in FIG. 2, a subroutine corresponding to a body temperature correction process based on a result of diagnosis. To distinguish the body temperature correction process based on a result of diagnosis from body temperature correction processes based on other types of patient condition information, the step of the body temperature correction process of FIG. 10 is represented by a reference number S102D.

At step S141, the processor 11 obtains a result of diagnosis of a disease of the patient 100 performed by a doctor. The processor 11 may read a pre-stored result of diagnosis from the storage device 13, may obtain a result of diagnosis via the input device 15, or may obtain a result of diagnosis from an external server apparatus (not shown) via the communication device 14.

At step S142, the processor 11 corrects the current body temperature of the patient 100 based on the result of diagnosis. A corrected body temperature Td may be calculated by, for example, Td=T×k1 based on a current body temperature T and a coefficient k1 predetermined for each disease. Examples of coefficients k1 are listed below.
Covid-19 (new coronavirus) 1
SARS 1
Norovirus infection 1.05
Common cold (colds other than those listed above) 0.98
Hepatitis A, hepatitis C 1.03

The corrected body temperature Td may be calculated by, for example, Td=T+k2 based on the current body temperature T and a constant k2 predetermined for each disease. For example, in the case of seasonal influenza, the constant k2 may be set to +0.5.

Performing the body temperature correction process based on a result of diagnosis makes it possible to reduce the influence of various types of diseases and thereby makes it possible to calculate a risk related to the health status of the patient 100 more accurately than the related art.

### Body Temperature Correction Process Based on Pulse

Generally, when the body temperature of a patient increases, the pulse of the patient also increases. However, when a patient has a certain type of infectious disease, the pulse does not increase much even if the body temperature increases. This state is called “relative bradycardia”. When relative bradycardia is present, the health status of the patient is considered to be at a higher risk than in a case in which relative bradycardia is not present. Below, a method of correcting the body temperature of a patient taking into account the influence of relative bradycardia is described.

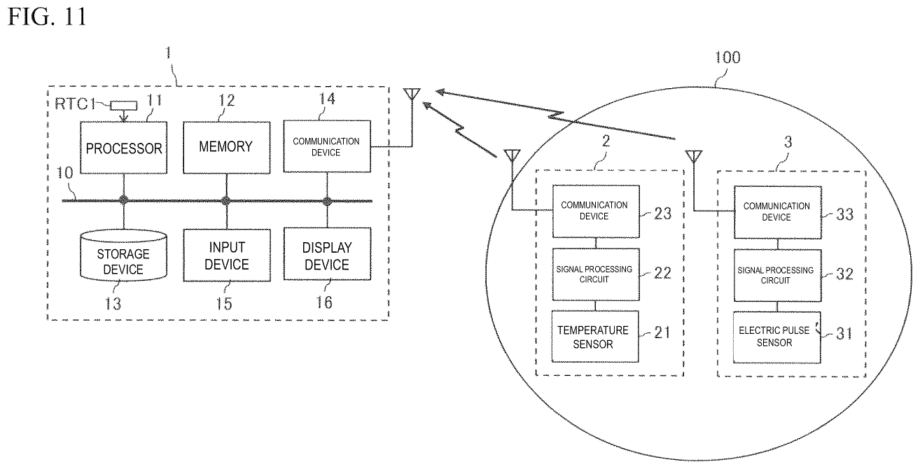

FIG. 11 is a block diagram illustrating a configuration of a risk calculation system according to a variation of the first embodiment. In addition to the risk calculation apparatus 1 and the thermometer 2 in the risk calculation system of FIG. 1, the risk calculation system of FIG. 11 includes a pulse meter 3 attached to the body of the patient 100.

The risk calculation apparatus 1 in FIG. 11 obtains the body temperature of the patient 100 from the thermometer 2, obtains the pulse of the patient 100 from the pulse meter 3, and calculates a risk related to the health status of the patient 100 based on the body temperature and the pulse of the patient 100. The risk calculation apparatus 1 in FIG. 11 has substantially the same configuration as the risk calculation apparatus 1 in FIG. 1 except that the risk calculation apparatus 1 in FIG. 11 is connected to the pulse meter 3 for communication and performs a risk calculation process 102E described later.

The pulse meter 3 includes an electric pulse sensor 31, a signal processing circuit 32, and a communication device 33. The electric pulse sensor 31 detects the pulse of the patient 100. The signal processing circuit 32 converts the pulse of the patient 100 detected by the electric pulse sensor 31 into a format (for example, a digital value) that can be transmitted to the risk calculation apparatus 1. The communication device 33 is connected for communication to the risk calculation apparatus 1 and transmits the pulse of the patient 100 to the risk calculation apparatus 1.

The pulse meter 3 may be a dedicated device for measuring the pulse or may be any other device, such as a pulse oximeter, an activity meter, a fatigue meter, or a carbohydrate meter, that has a function to measure the pulse. The pulse meter 3 may be wirelessly connected to the risk calculation apparatus 1 via Bluetooth (registered trademark) or WiFi (registered trademark), or may be connected by wire to the risk calculation apparatus 1. The pulse meter 3 may be provided separately from the thermometer 2 or may be integrated with the thermometer 2.

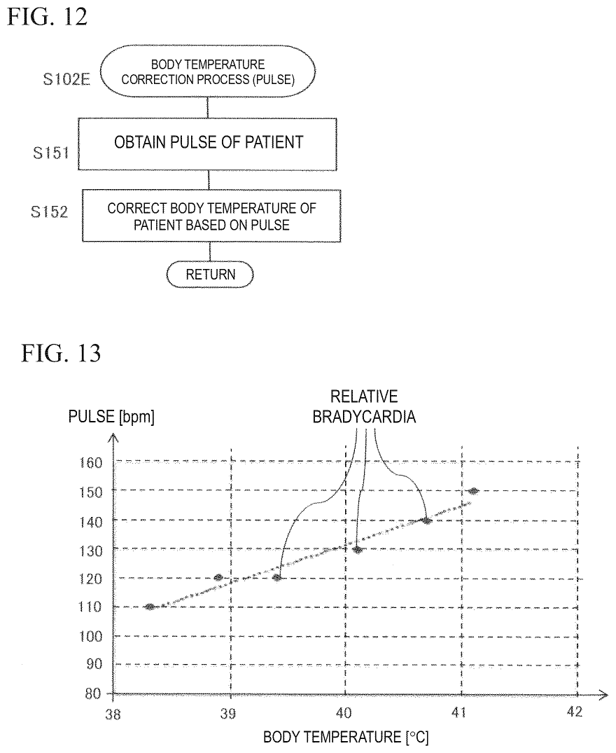

FIG. 12 is a flowchart illustrating, as a fifth example of step S102 in FIG. 2, a subroutine corresponding to a body temperature correction process based on a pulse. To distinguish the body temperature correction process based on a pulse from body temperature correction processes based on other types of patient condition information, the step of the body temperature correction process of FIG. 12 is represented by a reference number S102E. The processor 11 in FIG. 11 performs the risk calculation process of FIG. 2 and at step S102 in FIG. 2, performs the body temperature correction process of FIG. 12.

At step S151, the processor 11 obtains a measured pulse of the patient 100 from the pulse meter 3 via the communication device 14.

At step S152, the processor 11 calculates relative bradycardia based on the pulse and corrects the current body temperature of the patient 100 based on the relative bradycardia. Here, “corrects the current body temperature of the patient 100 based on the relative bradycardia” includes correcting the current body temperature of the patient 100 such that the corrected body temperature follows the difference between a first body temperature increase actually measured and a second body temperature increase estimated based on the pulse. A corrected body temperature Te is calculated by a formula below based on the current body temperature T and a pulse p.

$$Te = T + k 3 \times ( measured body temperature increase - body temperature increase \\ estimated based on pulse ) = T + k 3 \times ( ( T - Tm ) - k 4 \times ( p - pm ) ) .$$

Tm indicates a normal body temperature, and pm indicates a normal pulse. Also, k3 is a constant determined based on clinical practice and is set to, for example, 0.2. For example, when the pulse increases by 10 every time when the body temperature increases by 1 degree, k4 is set to 0.1.

For example, when a patient P1, who has a normal body temperature Tm1=36.5 degrees and a normal pulse pm1=60 bpm, has a measured body temperature T1=38.5 degrees and a measured pulse p1=80 bpm, a corrected body temperature Te1 is calculated by a formula below.

$$Te 1 = 38 \text{.5} + 0.2 \times ( 38.5 - 36.5 ) - 0.1 \times ( 80 - 60 ) ) = 38.5 [ ° C . ]$$

In this case, the body temperature increase estimated based on the pulse p1 is 0.1×(80−60)=2 degrees, and the measured body temperature T1 agrees with the estimated value.

Also, when a patient P2, who has a normal body temperature Tm2=36.8 degrees and a normal pulse pm2=60 bpm, has a measured body temperature T2=38.8 degrees and a measured pulse p2=65 bpm, a corrected body temperature Te2 is calculated by a formula below.

$$Te 2 = 38.8 + 0.2 \times ( 38.8 - 36.8 ) - 0.1 \times ( 65 - 60 ) ) = 39.1 [ ° C . ]$$

In this case, although the body temperature increase estimated based on the pulse p2 is 0.1×(65−60)=0.5 degrees, the measured body temperature increase is 2 degrees. Accordingly, the patient P2 is assumed to be in a state of relative bradycardia. Taking into account the risk of relative bradycardia, the corrected body temperature Te2 of the patient P2 is increased by 0.3 degrees from the measured body temperature T2.

While the body temperature increases of the patients P1 and P2 from their normal body temperatures are substantially the same, the pulse of the patient P2 does not substantially increase from the normal pulse. This is considered to be a risk of the worsening of the disease of the patient P2. Accordingly, the current body temperature of the patient P2 is increased by the correction.

FIG. 13 is a graph used to describe the occurrence of relative bradycardia. Dots below a thick dashed line indicate relative bradycardia. As the pulse located below the thick dashed line decreases, the severity of relative bradycardia increases.

According to the above formula for obtaining the corrected body temperature Te, when the body temperature is the same, the corrected body temperature Te increases as the pulse decreases. Thus, the risk attributable to relative bradycardia is reflected in the finally determined risk related to the health status of the patient 100.

Performing the body temperature correction process based on a pulse makes it possible to reduce the influence of the relationship between the pulse increase and the body temperature increase of the patient and thereby makes it possible to calculate a risk related to the health status of the patient 100 more accurately than the related art.

Because the influence of the relationship between the pulse increase and the body temperature increase is reduced, it is possible to effectively correct the body temperature even when the pulse is influenced by the difference between home care and hospital treatment or by the presence or absence of oxygen inhalation.

As described above, performing the risk calculation process including the body temperature correction process makes it possible to calculate a risk related to the health status of the patient 100 more accurately than the related art regardless of the condition of the patient 100.

The processor 11 may perform a combination of two or more of the body temperature correction process based on a circadian rhythm, the body temperature correction process based on a basal metabolic rate, the body temperature correction process based on medication, the body temperature correction process based on a result of diagnosis, and the body temperature correction process based on a pulse. This makes it possible to more accurately calculate a risk related to the health status of the patient 100.

Instead of correcting a measured current body temperature of the patient 100, a set value may be corrected as an equivalent method. Next, a risk calculation process including a set value correction process is described.

### Risk Calculation Process Including Set Value Correction Process

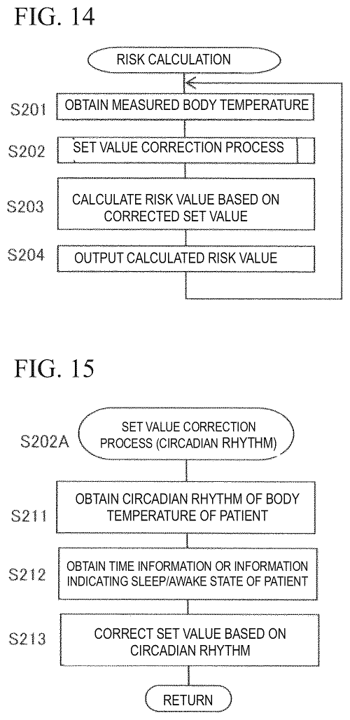

FIG. 14 is a flowchart illustrating a second example of a risk calculation process that is performed by the processor 11 illustrated in FIG. 1 and includes a set value correction process. According to the process of FIG. 14, the processor 11 corrects (i.e., sets or resets) a set value before calculating a risk value.

At step S201, the processor 11 obtains a measured current body temperature of the patient 100 from the thermometer 2 via the communication device 14.

At step S202, the processor 11 performs the set value correction process to correct the set value based on patient condition information.

At step S203, the processor 11 calculates a risk value based on the measured body temperature and the corrected set value.

At step S204, the processor 11 outputs the calculated risk value to the display device 16.

The processor 11 periodically repeats the risk calculation process of FIG. 14.

As described above, the patient condition information includes, for example, at least one of a circadian rhythm of the body temperature of the patient 100, a basal metabolic rate of the patient 100 or information associated the basal metabolic rate, medication information about a drug administered to the patient 100, a result of diagnosis of a disease of the patient 100, and a pulse of the patient 100. Below, a set value correction process based on a circadian rhythm, a set value correction process based on a basal metabolic rate, a set value correction process based on medication, a set value correction process based on a result of diagnosis, and a set value correction process based on a pulse are described.

### Set Value Correction Process Based on Circadian Rhythm

FIG. 15 is a flowchart illustrating, as a first example of step S202 in FIG. 14, a subroutine corresponding to a set value correction process based on a circadian rhythm. To distinguish the set value correction process based on a circadian rhythm from set value correction processes based on other types of patient condition information, the step of the set value correction process of FIG. 15 is represented by a reference number S202A.

At step S211, the processor 11 reads the circadian rhythm of the body temperature of the patient 100 from the storage device 13.

At step S212, the processor 11 obtains time information indicating the current time from the clock RTC1 or obtains activity information indicating whether the patient 100 is currently asleep or awake via the input device 15.

Steps S211 and S212 are the same as steps S111 and S112 in FIG. 3.

At step S213, the processor 11 corrects a set value based on the circadian rhythm and the current time, or the activity information, such that the corrected set value follows the variation in the body temperature of the patient 100 according to the circadian rhythm. Here, “follows the variation in the body temperature” indicates that the set value at least partially follows the increase or decrease in the body temperature according to the circadian rhythm.

Performing the set value correction process based on the circadian rhythm makes it possible to reduce the influence of the circadian variation in the body temperature of the patient 100 and thereby makes it possible to calculate a risk related to the health status of the patient 100 more accurately than the related art regardless of the time of day.

### Set Value Correction Process Based on Basal Metabolic Rate

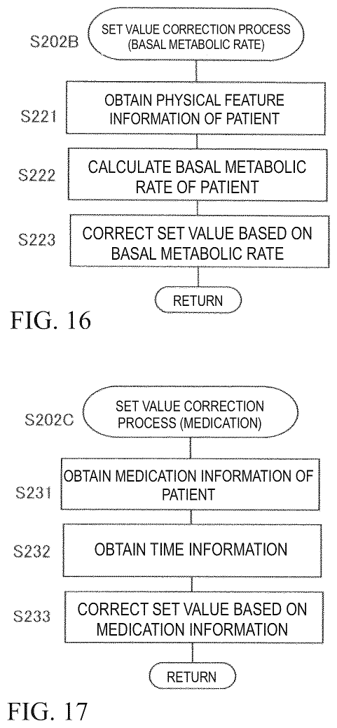

FIG. 16 is a flowchart illustrating, as a second example of step S202 in FIG. 2, a subroutine corresponding to a set value correction process based on a basal metabolic rate. To distinguish the set value correction process based on the basal metabolic rate from set value correction processes based on other types of patient condition information, the step of the set value correction process of FIG. 16 is represented by a reference number S202B.

At step S221, the processor 11 obtains physical feature information of the patient 100.

At step S222, the processor 11 calculates the basal metabolic rate of the patient 100 based on the physical feature information of the patient 100.

Steps S221 and S222 are the same as steps S121 and S122 in FIG. 6.

At step S223, the processor 11 corrects the set value based on the basal metabolic rate such that the corrected set value follows the increase or decrease in the basal metabolic rate. Here, “follows the increase or decrease in the basal metabolic rate” indicates that the set value at least partially follows the increase or decrease in the body temperature due to the individual variation in the basal metabolic rate.

Performing the set value correction process based on the basal metabolic rate makes it possible to reduce the influence of the individual variation in the basal metabolic rate and thereby makes it possible to calculate a risk related to the health status of the patient 100 more accurately than the related art.

### Set Value Correction Process Based on Medication

FIG. 17 is a flowchart illustrating, as a third example of step S202 in FIG. 2, a subroutine corresponding to a set value correction process based on medication. To distinguish the set value correction process based on the medication from set value correction processes based on other types of patient condition information, the step of the set value correction process of FIG. 17 is represented by a reference number S202C.

At step S231, the processor 11 obtains medication information regarding a drug administered to the patient 100.

At step S232, the processor 11 obtains time information indicating the current time from the clock RTC1.

Steps S231 and S232 are the same as steps S131 and S132 in FIG. 7.

At step S233, the processor 11 corrects a set value based on the medication information such that the corrected set value follows the increase or decrease in the body temperature of the patient 100 caused by the drug. Here, “follows the increase or decrease in the body temperature of the patient 100 caused by the drug” indicates that the set value at least partially follows the increase or decrease in the body temperature caused by the drug.

Performing the set value correction process based on the medication makes it possible to reduce the influence of the drug administered to the patient 100 and thereby makes it possible to calculate a risk related to the health status of the patient 100 more accurately than the related art.

### Set Value Correction Process Based on Result of Diagnosis

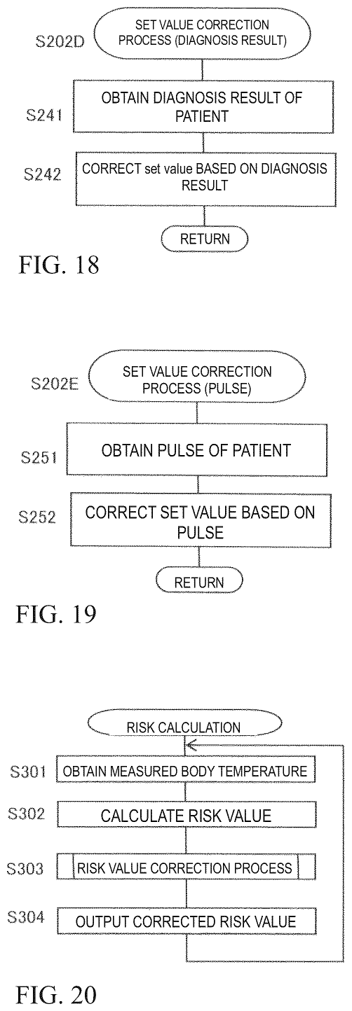

FIG. 18 is a flowchart illustrating, as a fourth example of step S202 in FIG. 2, a subroutine corresponding to a set value correction process based on a result of diagnosis. To distinguish the set value correction process based on the result of diagnosis from set value correction processes based on other types of patient condition information, the step of the set value correction process of FIG. 18 is represented by a reference number S202D.

At step S241, the processor 11 obtains a result of diagnosis of a disease of the patient 100 performed by a doctor. Step S241 is the same as step S114 in FIG. 10.

At step S242, the processor 11 corrects the set value based on the result of diagnosis. The corrected set value is calculated by multiplying an original set value by a coefficient predetermined for each disease or by adding or subtracting a constant predetermined for each disease to or from the original set value.

Performing the set value correction process based on the result of diagnosis makes it possible to reduce the influence of various types of diseases and thereby makes it possible to calculate a risk related to the health status of the patient 100 more accurately than the related art.

### Set Value Correction Process Based on Pulse

FIG. 19 is a flowchart illustrating, as a fifth example of step S202 in FIG. 2, a subroutine corresponding to a set value correction process based on a pulse. To distinguish the set value correction process based on the pulse from set value correction processes based on other types of patient condition information, the step of the set value correction process of FIG. 19 is represented by a reference number S202E.

At step S251, the processor 11 obtains a measured pulse of the patient 100 from the pulse meter 3 via the communication device 14.

Step S251 is the same as step S151 in FIG. 12.

At step S252, the processor 11 corrects the set value of the patient 100 based on the pulse to offset the difference between a first body temperature increase actually measured and a second body temperature increase estimated based on the pulse.

Performing the set value correction process based on the pulse makes it possible to calculate a risk related to the health status of the patient 100 more accurately than the related art by taking into account the influence of relative bradycardia.

As described above, performing the risk calculation process including the set value correction process makes it possible to calculate a risk related to the health status of the patient 100 more accurately than the related art regardless of the condition of the patient 100.

The processor 11 may perform a combination of two or more of the set value correction process based on a circadian rhythm, the set value correction process based on a basal metabolic rate, the set value correction process based on medication, the set value correction process based on a result of diagnosis, and the set value correction process based on a pulse. This makes it possible to more accurately calculate a risk related to the health status of the patient 100.

Instead of correcting the measured current body temperature of the patient 100, a calculated risk value may be corrected as an equivalent method. Next, a risk calculation process including a risk value correction process is described.

### Risk Calculation Process Including Risk Value Correction Process

FIG. 20 is a flowchart illustrating a third example of a risk calculation process that is performed by the processor 11 illustrated in FIG. 1 and includes a risk value correction process. According to the process of FIG. 20, the processor 11 corrects (i.e., increases or decreases) a risk value after calculating the risk value.

At step S301, the processor 11 obtains a measured current body temperature of the patient 100 from the thermometer 2 via the communication device 14.

At step S302, the processor 11 calculates a risk value based on the measured body temperature and a set value.

At step S303, the processor 11 performs the risk value correction process to correct the risk value based on patient condition information.

At step S304, the processor 11 outputs the corrected risk value to the display device 16.

The processor 11 periodically repeats the risk calculation process of FIG. 20.

As described above, the patient condition information includes, for example, at least one of a circadian rhythm of the body temperature of the patient 100, a basal metabolic rate of the patient 100 or information associated the basal metabolic rate, medication information about a drug administered to the patient 100, a result of diagnosis of a disease of the patient 100, and a pulse of the patient 100. Below, a risk value correction process based on a circadian rhythm, a risk value correction process based on a basal metabolic rate, a risk value correction process based on medication, a risk value correction process based on a result of diagnosis, and a risk value correction process based on a pulse are described.

### Risk Value Correction Process Based on Circadian Rhythm

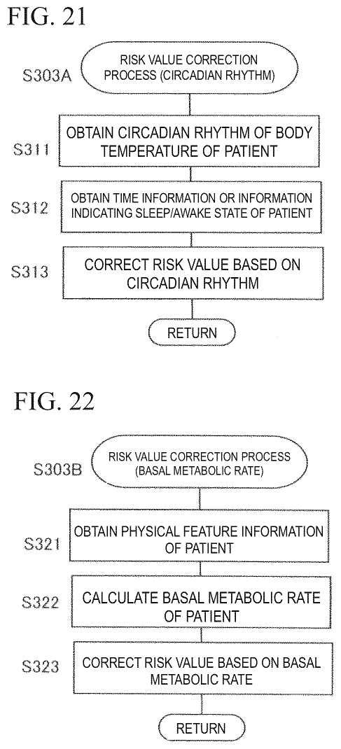

FIG. 21 is a flowchart illustrating, as a first example of step S303 in FIG. 20, a subroutine corresponding to a risk value correction process based on a circadian rhythm. To distinguish the risk value correction process based on the circadian rhythm from risk value correction processes based on other types of patient condition information, the step of the risk value correction process of FIG. 21 is represented by a reference number S303A.

At step S311, the processor 11 reads the circadian rhythm of the body temperature of the patient 100 from the storage device 13.

At step S312, the processor 11 obtains time information indicating the current time from the clock RTC1 or obtains activity information indicating whether the patient 100 is currently asleep or awake via the input device 15.

Steps S311 and S312 are the same as steps S111 and S112 in FIG. 3.

At step S313, the processor 11 corrects the calculated risk value based on the circadian rhythm and the current time, or the activity information, to offset the variation in the body temperature of the patient 100 according to the circadian rhythm. Here, “offset the variation in the body temperature” indicates at least partially offsetting the increase or decrease in the body temperature according to the circadian rhythm.

Performing the risk value correction process based on the circadian rhythm makes it possible to reduce the influence of the circadian variation in the body temperature of the patient 100 and thereby makes it possible to calculate a risk related to the health status of the patient 100 more accurately than the related art regardless of the time of day.

### Risk Value Correction Process Based on Basal Metabolic Rate

FIG. 22 is a flowchart illustrating, as a second example of step S303 in FIG. 20, a subroutine corresponding to a risk value correction process based on a basal metabolic rate. To distinguish the risk value correction process based on the basal metabolic rate from risk value correction processes based on other types of patient condition information, the step of the risk value correction process of FIG. 22 is represented by a reference number S303B.

At step S321, the processor 11 obtains physical feature information of the patient 100.

At step S322, the processor 11 calculates the basal metabolic rate of the patient 100 based on the physical feature information of the patient 100.

Steps S321 and S322 are the same as steps S121 and S122 in FIG. 6.

At step S323, the processor 11 corrects the calculated risk value based on the basal metabolic rate to offset the increase or decrease in the basal metabolic rate. Here, “offset the increase or decrease in the basal metabolic rate” indicates at least partially offsetting the increase or decrease in the body temperature due to the individual variation in the basal metabolic rate.

Performing the risk value correction process based on the basal metabolic rate makes it possible to reduce the influence of the individual variation in the basal metabolic rate and thereby makes it possible to calculate a risk related to the health status of the patient 100 more accurately than the related art.

### Risk Value Correction Process Based on Medication

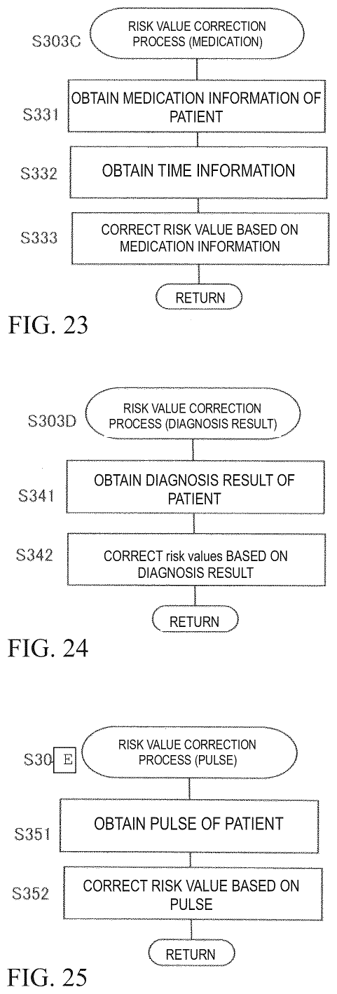

FIG. 23 is a flowchart illustrating, as a third example of step S303 in FIG. 20, a subroutine corresponding to a risk value correction process based on medication. To distinguish the risk value correction process based on the medication from risk value correction processes based on other types of patient condition information, the step of the risk value correction process of FIG. 23 is represented by a reference number S303C.

At step S331, the processor 11 obtains medication information regarding a drug administered to the patient 100.

At step S332, the processor 11 obtains time information indicating the current time from the clock RTC1.

Steps S331 and S332 are the same as steps S131 and S132 in FIG. 7.

At step S333, the processor 11 corrects the calculated risk value based on the medication information to offset the increase or decrease in the body temperature of the patient 100 caused by the drug. Here, “offset the increase or decrease in the body temperature of the patient 100 caused by the drug” indicates at least partially offsetting the increase or decrease in the body temperature caused by the drug.

Performing the risk value correction process based on the medication makes it possible to reduce the influence of the drug administered to the patient 100 and thereby makes it possible to calculate a risk related to the health status of the patient 100 more accurately than the related art.

### Risk Value Correction Process Based on Result of Diagnosis

FIG. 24 is a flowchart illustrating, as a fourth example of step S303 in FIG. 20, a subroutine corresponding to a risk value correction process based on a result of diagnosis. To distinguish the risk value correction process based on the result of diagnosis from risk value correction processes based on other types of patient condition information, the step of the risk value correction process of FIG. 24 is represented by a reference number S303D.

At step S341, the processor 11 obtains a result of diagnosis of a disease of the patient 100 performed by a doctor. Step S341 is the same as step S114 in FIG. 10.

At step S342, the processor 11 corrects the calculated risk value based on the result of diagnosis. The corrected risk value is calculated by multiplying the originally calculated risk value by a coefficient predetermined for each disease or by adding or subtracting a constant predetermined for each disease to or from the originally calculated risk value.

Performing the risk value correction process based on the result of diagnosis makes it possible to reduce the influence of various types of diseases and thereby makes it possible to calculate a risk related to the health status of the patient 100 more accurately than the related art.

### Risk Value Correction Process Based on Pulse

FIG. 25 is a flowchart illustrating, as a fifth example of step S303 in FIG. 20, a subroutine corresponding to a risk value correction process based on a pulse. To distinguish the risk value correction process based on the pulse from risk value correction processes based on other types of patient condition information, the step of the risk value correction process of FIG. 25 is represented by a reference number S303E.

At step S351, the processor 11 obtains a measured pulse of the patient 100 from the pulse meter 3 via the communication device 14.

Step S351 is the same as step S151 in FIG. 12.

At step S352, the processor 11 corrects the calculated risk value based on the pulse such that the corrected risk value follows the difference between a first body temperature increase actually measured and a second body temperature increase estimated based on the pulse.

Performing the risk value correction process based on the pulse makes it possible to calculate a risk related to the health status of the patient 100 more accurately than the related art by taking into account the influence of relative bradycardia.

As described above, performing the risk calculation process including the risk value correction process makes it possible to calculate a risk related to the health status of the patient 100 more accurately than the related art regardless of the condition of the patient 100.

The processor 11 may perform a combination of two or more of the risk value correction process based on a circadian rhythm, the risk value correction process based on a basal metabolic rate, the risk value correction process based on medication, the risk value correction process based on a result of diagnosis, and the risk value correction process based on a pulse. This makes it possible to more accurately calculate a risk related to the health status of the patient 100.

### Summary of First Embodiment

The storage device 13 of the risk calculation apparatus 1 of FIG. 1 stores a program including instructions executable by a processor of a computer to calculate a risk related to the health status of the patient 100. The instructions cause the processor to perform a first step of obtaining a body temperature of the patient 100 and patient 100 condition information and a second step of comparing the body temperature of the patient 100 with a reference temperature to calculate a comparison result and calculating a risk related to the health status of the patient 100 based on the comparison result. The second step includes at least one of a process of correcting the body temperature based on the patient 100 condition information when comparing the body temperature with the reference temperature, a process of setting the reference temperature based on the patient 100 condition information when comparing the body temperature with the reference temperature, and a process of increasing or decreasing the comparison result based on the patient 100 condition information.

As described above, parameters measured to calculate a risk related to the health status of the patient 100 may vary due to factors, such as a circadian rhythm, a basal metabolic rate, and medication, that are not directly related to a disease of the patient. This may lead to a problem in which the risk related to the health status of the patient 100 is calculated incorrectly. Furthermore, even when the measured value of a parameter is the same, the magnitude of the risk varies depending on the type of disease that a patient has or is suspected to have. The risk calculation apparatus 1 according to the embodiment can accurately calculate a risk related to the health status of the patient 100 in real time regardless of the condition of the patient 100 by correcting a current body temperature, a temperature threshold, or a risk value based on the patient condition information. Accordingly, signs of the onset or worsening of a disease can always be accurately detected.

Conventionally, a measured body temperature is corrected by a doctor based on empirical knowledge. The risk calculation apparatus 1 according to the embodiment can automatically correct the current body temperature, the temperature threshold, or the risk value and can therefore reduce the time and effort of healthcare workers.

### Second Embodiment

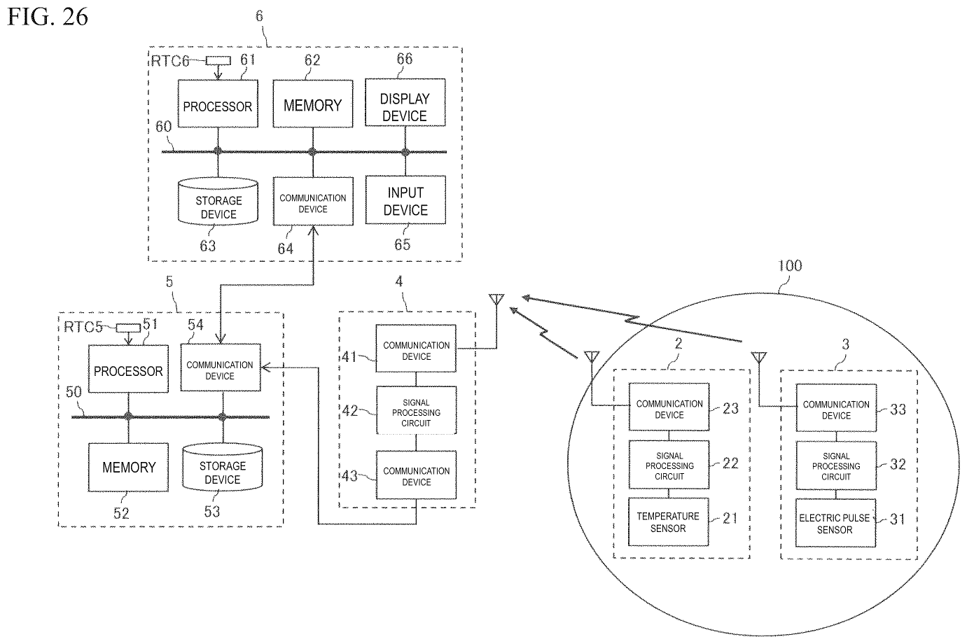

FIG. 26 is a block diagram illustrating a configuration of a risk calculation system according to a second embodiment. The risk calculation system of FIG. 26 includes a thermometer 2, a pulse meter 3, a gateway apparatus 4, a server apparatus 5, and a client apparatus 6.

As with the thermometer 2 and the pulse meter 3 in FIG. 11, the thermometer 2 and the pulse meter 3 in FIG. 26 are attached to the body of the patient 100 and detect the body temperature and the pulse of the patient 100, respectively.

The gateway apparatus 4 includes a communication device 41, a signal processing circuit 42, and a communication device 43. The communication device 41 is connected to the thermometer 2 and the pulse meter 3 for communication, receives the body temperature of the patient 100 from the thermometer 2, and receives the pulse of the patient 100 from the pulse meter 3. The signal processing circuit 42 converts the received body temperature and pulse into formats that can be transmitted to the server apparatus 5. The communication device 43 is connected to the server apparatus 5 for communication and transmits the body temperature and the pulse to the server apparatus 5.

The gateway apparatus 4 may also be connected for communication to other devices, such as a pulse oximeter, an activity meter, a fatigue meter, and a glucometer, that obtain parameters indicating conditions of the patient 100 and may transmit the parameters obtained from those devices to the server apparatus 5. The gateway apparatus 4 may be wirelessly connected to the server apparatus 5 via, for example, LTE or may be connected by wire to the server apparatus 5.

The server apparatus 5 includes a bus 50, a processor 51, a memory 52, a storage device 53, a communication device 54, and a clock RTC5. The processor 51 controls the operation of the entire server apparatus 5. The memory 52 temporarily stores programs and data necessary for the operation of the server apparatus 5. The storage device 53 is a non-volatile storage medium that stores programs and data necessary for the operation of the server apparatus 5. The communication device 54 is connected to the gateway apparatus 4 for communication and obtains the body temperature and the pulse of the patient 500 from the gateway apparatus 4. The communication device 54 is connected to the client apparatus 6 for communication. The clock RTC5 provides time information indicating the current time. The processor 51, the memory 52, the storage device 53, and the communication device 54 are connected to each other via the bus 50.

The client apparatus 6 includes a bus 60, a processor 61, a memory 62, a storage device 63, a communication device 64, an input device 65, a display device 66, and a clock RTC6. The processor 61 controls the operation of the entire client apparatus 6. The memory 62 temporarily stores programs and data necessary for the operation of the client apparatus 6. The storage device 63 is a non-volatile storage medium that stores programs and data necessary for the operation of the client apparatus 6. The communication device 64 is connected to the server apparatus 5 for communication. The input device 65 receives user inputs for controlling the operation of the client apparatus 6. The input device 65 includes, for example, a keyboard and a pointing device. The display device 66 displays a calculated risk related to the health status of the patient 500. The clock RTC5 provides time information indicating the current time. The processor 61, the memory 62, the storage device 63, the communication device 64, the input device 65, and the display device 66 are connected to each other via the bus 60.

In the risk calculation system of FIG. 26, the processor 51 of the server apparatus 5 may perform the risk calculation process of FIG. 2, FIG. 14, or FIG. 20, and the client apparatus 6 may obtain a calculated risk related to the health status of the patient 500 from the server apparatus 5 and display the obtained risk on the display device 66. Alternatively, the server apparatus 5 may temporarily store the measured body temperature and pulse and the patient condition information of the patient 100 in the storage device 53. In this case, the processor 61 of the client apparatus 6 obtains the body temperature, the pulse, and the patient condition information of the patient 100 from the server apparatus 5, performs the risk calculation process of FIG. 2, FIG. 14, or FIG. 20, and displays a calculated risk related to the health status of the patient 500 on the display device 66. The storage device 53 of the server apparatus 5 or the storage device 63 of the client apparatus 6 stores a computer-executable program for calculating a risk related to the health status of the patient 100.

According to the second embodiment, the risk calculation system can be constructed with a high degree of flexibility.

### Other Variations

The processor 11 may perform a combination of two or three of the risk calculation process including the body temperature correction process, the risk calculation process including the set value correction process, and the risk calculation process including the risk value correction process.

### Summary of Embodiments

A risk calculation apparatus, a risk calculation system, and a program according to various aspects of this disclosure may have configurations described below.

A risk calculation apparatus according to a first aspect calculates a risk related to the health status of a patient. The risk calculation apparatus includes an input unit that obtains a body temperature and patient condition information of the patient, and a calculation unit that calculates a comparison result by comparing the body temperature obtained by the input unit with a reference temperature and outputs the risk related to the health status of the patient based on the comparison result. The calculation unit performs at least one of a process of correcting the body temperature based on the patient condition information when comparing the body temperature with the reference temperature, a process of setting the reference temperature based on the patient condition information when comparing the body temperature with the reference temperature, and a process of increasing or decreasing the comparison result based on the patient condition information.

According to a second aspect, in the risk calculation apparatus according to the first aspect, the patient condition information includes variation according to a circadian rhythm in the body temperature of the patient; and the calculation unit performs, based on the variation according to the circadian rhythm, at least one of the process of correcting the body temperature, the process of setting the reference temperature, and the process of increasing or decreasing the comparison result.

According to a third aspect, in the risk calculation apparatus according to the second aspect, the calculation unit performs at least one of the process of correcting the body temperature, the process of setting the reference temperature, and the process of increasing or decreasing the comparison result to offset the variation according to the circadian rhythm.

According to a fourth aspect, in the risk calculation apparatus according to the second aspect, the calculation unit performs at least one of the process of correcting the body temperature, the process of setting the reference temperature, and the process of increasing or decreasing the comparison result to follow the variation according to the circadian rhythm.

According to a fifth aspect, in the risk calculation apparatus according to any one of the second through fourth aspects, the input unit continuously obtains the patient condition information; and the calculation unit performs, based on a time when the input unit obtains the body temperature of the patient, at least one of the process of correcting the body temperature, the process of setting the reference temperature, and the process of increasing or decreasing the comparison result.

According to a sixth aspect, in the risk calculation apparatus according to any one of the second through fourth aspects, the patient condition information includes activity information indicating whether the patient is currently asleep or awake; and the calculation unit performs, based on the activity information, at least one of the process of correcting the body temperature, the process of setting the reference temperature, and the process of increasing or decreasing the comparison result.

According to a seventh aspect, in the risk calculation apparatus according to any one of the first through sixth aspects, the patient condition information includes a basal metabolic rate of the patient or information for calculating the basal metabolic rate of the patient; and the calculation unit performs, based on the basal metabolic rate of the patient and a standard basal metabolism, at least one of the process of correcting the body temperature, the process of setting the reference temperature, and the process of increasing or decreasing the comparison result.

According to an eighth aspect, in the risk calculation apparatus according to the seventh aspect, the calculation unit performs at least one of the process of correcting the body temperature, the process of setting the reference temperature, and the process of increasing or decreasing the comparison result to offset an increase or decrease in the basal metabolic rate of the patient from the standard basal metabolism.

According to a ninth aspect, in the risk calculation apparatus according to the seventh aspect, the calculation unit performs at least one of the process of correcting the body temperature, the process of setting the reference temperature, and the process of increasing or decreasing the comparison result to follow an increase or decrease in the basal metabolic rate of the patient from the standard basal metabolism.

According to a tenth aspect, in the risk calculation apparatus according to any one of the first through ninth aspects, the patient condition information includes medication information indicating a drug administered to the patient; and the calculation unit performs, based on the medication information, at least one of the process of correcting the body temperature, the process of setting the reference temperature, and the process of increasing or decreasing the comparison result.

According to an eleventh aspect, in the risk calculation apparatus according to the tenth aspect, the calculation unit performs at least one of the process of correcting the body temperature, the process of setting the reference temperature, and the process of increasing or decreasing the comparison result to offset an increase or decrease in the body temperature of the patient caused by the drug.

According to a twelfth aspect, in the risk calculation apparatus according to the tenth aspect, the calculation unit performs at least one of the process of correcting the body temperature, the process of setting the reference temperature, and the process of increasing or decreasing the comparison result to follow an increase or decrease in the body temperature of the patient caused by the drug.

According to a thirteenth aspect, in the risk calculation apparatus according to any one of the tenth through twelfth aspects, the medication information includes at least one of a type of the drug, elapsed time after the drug is administered, and an administration method of the drug.

According to a fourteenth aspect, in the risk calculation apparatus according to any one of the first through thirteenth aspects, the patient condition information includes a result of diagnosis of a disease of the patient; and the calculation unit performs, based on the result of diagnosis, at least one of the process of correcting the body temperature, the process of setting the reference temperature, and the process of increasing or decreasing the comparison result.

According to a fifteenth aspect, in the risk calculation apparatus according to any one of the first through fourteenth aspects, the patient condition information includes a pulse of the patient; and the calculation unit calculates relative bradycardia based on the pulse and performs, based on the relative bradycardia, at least one of the process of correcting the body temperature, the process of setting the reference temperature, and the process of increasing or decreasing the comparison result.

According to a sixteenth aspect, in the risk calculation apparatus according to the fifteenth aspect, the calculation unit performs at least one of the process of correcting the body temperature, the process of setting the reference temperature, and the process of increasing or decreasing the comparison result to follow a difference between a first body temperature increase actually measured and a second body temperature increase estimated based on the pulse.

According to a seventeenth aspect, in the risk calculation apparatus according to the fifteenth aspect, the calculation unit performs at least one of the process of correcting the body temperature, the process of setting the reference temperature, and the process of increasing or decreasing the comparison result to offset a difference between a first body temperature increase actually measured and a second body temperature increase estimated based on the pulse.

A risk calculation system according to an eighteenth aspect includes a temperature sensor that measures the body temperature of the patient and the risk calculation apparatus according to any one of the first through seventeenth aspects.

A program according to a nineteenth aspect includes instructions that are executable by a processor of a computer to calculate a risk related to the health status of a patient. The instructions cause the processor to perform a first step of obtaining a body temperature and patient condition information of the patient, and a second step of comparing the body temperature of the patient with a reference temperature to calculate a comparison result and calculating the risk related to the health status of the patient based on the comparison result. The second step includes at least one of a process of correcting the body temperature based on the patient condition information when comparing the body temperature with the reference temperature, a process of setting the reference temperature based on the patient condition information when comparing the body temperature with the reference temperature, and a process of increasing or decreasing the comparison result based on the patient condition information.

A risk calculation apparatus, a risk calculation system, and a program according to an aspect of the present disclosure can be used to calculate a risk related to the health status of a patient.
1 risk calculation apparatus
2 thermometer
3 pulse meter
4 gateway apparatus
5 server apparatus
6 client apparatus
10 bus
11 processor
12 memory
13 storage device
14 communication device
15 input device
16 display device
21 temperature sensor
22 signal processing circuit
23 communication device
31 electric pulse sensor
32 signal processing circuit
33 communication device
41 communication device
42 signal processing circuit
43 communication device
50 bus
51 processor
52 memory
53 storage device
54 communication device
60 bus
61 processor
62 memory
63 storage device
64 communication device
65 input device
66 display device
100 patient
RTC1, RTC5, RTC6 clock

1. A risk calculation apparatus configured to calculate a risk related to a health status of a patient, the risk calculation apparatus comprising:
at least one processor and memory collectively configured to:
obtain a body temperature and patient condition information of the patient; and
compare the obtained body temperature with a reference temperature;
correct the body temperature based on the patient condition information when comparing the body temperature with the reference temperature, set the reference temperature based on the patient condition information when comparing the body temperature with the reference temperature, or increase or decrease the comparison result based on the patient condition information; and
output the risk related to the health status of the patient based on the corrected body temperature.

2. The risk calculation apparatus according to claim 1,
wherein the patient condition information varies according to a circadian rhythm in the body temperature of the patient, and
wherein the processor is configured to, based on the variation according to the circadian rhythm, correct the body temperature, sets the reference temperature, or increases or decreases the comparison result.

3. The risk calculation apparatus according to claim 2, wherein the processor is configured to correct the body temperature, set the reference temperature, or increase or decrease the comparison result in a manner that offsets the variation according to the circadian rhythm.

4. The risk calculation apparatus according to claim 2, wherein the processor is configured to correct the body temperature, set the reference temperature, or increase or decrease the comparison result such that the body temperature, the reference temperature, or the comparison result follows the variation due to the circadian rhythm.

5. The risk calculation apparatus according to claim 2,
wherein the processor is configured to continuously obtain the patient condition information, and
wherein the processor is configured to, based on a time when the body temperature of the patient is obtained, correct the body temperature, set the reference temperature, or increase or decrease the comparison result.

6. The risk calculation apparatus according to claim 2,
wherein the patient condition information includes activity information indicating whether the patient is currently asleep or awake, and
wherein the processor is configured to, based on the activity information, correct the body temperature, set the reference temperature, or increase or decrease the comparison result.

7. The risk calculation apparatus according to claim 1,
wherein the patient condition information includes a basal metabolic rate of the patient or information for calculating the basal metabolic rate of the patient, and
wherein the processor is configured to, based on the basal metabolic rate of the patient and a standard basal metabolism, correct the body temperature, set the reference temperature, or increase or decrease the comparison result.

8. The risk calculation apparatus according to claim 7, wherein the processor is configured to correct the body temperature, set the reference temperature, or increase or decrease the comparison result in a manner that offsets an increase or decrease in the basal metabolic rate of the patient from the standard basal metabolism.

9. The risk calculation apparatus according to claim 7, wherein the processor is configured to correct the body temperature, set the reference temperature, or increase or decrease the comparison result such that the body temperature, the reference temperature, or the comparison result follows an increase or decrease in the basal metabolic rate of the patient from the standard basal metabolism.

10. The risk calculation apparatus according to claim 1,
wherein the patient condition information includes medication information indicating a drug administered to the patient, and
wherein the processor is configured to, based on the medication information, correct the body temperature, set the reference temperature, or increase or decrease the comparison result.

11. The risk calculation apparatus according to claim 10, wherein the processor is configured to correct the body temperature, set the reference temperature, or increase or decrease the comparison result in a manner that offsets an increase or decrease in the body temperature of the patient caused by the drug.

12. The risk calculation apparatus according to claim 10, wherein the processor is configured to correct the body temperature, set the reference temperature, or increase or decrease the comparison result such that the body temperature, the reference temperature, or the comparison result follows an increase or decrease in the body temperature of the patient caused by the drug.

13. The risk calculation apparatus according to claim 10, wherein the medication information includes a type of the drug, elapsed time after the drug is administered, or an administration method of the drug.

14. The risk calculation apparatus according to claim 1,
wherein the patient condition information includes a result of a diagnosis of a disease of the patient, and
wherein the processor is configured to, based on the result of diagnosis, correct the body temperature, set the reference temperature, or increase or decrease the comparison result.

15. The risk calculation apparatus according to claim 1,
wherein the patient condition information includes a pulse of the patient, and
wherein the processor is configured to:
determine relative bradycardia based on the pulse, and
based on the relative bradycardia, correct the body temperature, set the reference temperature, or increase or decrease the comparison result.

16. The risk calculation apparatus according to claim 15, wherein processor is configured to correct the body temperature, set the reference temperature, or increase or decrease the comparison result such that the body temperature, the reference temperature, or the comparison result follows a difference between a first body temperature increase actually measured and a second body temperature increase estimated based on the pulse.

17. The risk calculation apparatus according to claim 15, wherein the processor is configured to correct the body temperature, set the reference temperature, or increase or decrease the comparison result in a manner that offsets a difference between a first body temperature increase actually measured and a second body temperature increase estimated based on the pulse.

18. A risk calculation system comprising:
the risk calculation apparatus according to claim 1; and
a temperature sensor configured to measure the body temperature of the patient.

19. A non-transitory computer-readable medium having instructions stored thereon that, when executed by a processor of a computer, cause the processor to calculate a risk related to a health status of a patient by:
obtaining a body temperature and patient condition information of the patient;
comparing the body temperature of the patient with a reference temperature;
correcting the body temperature based on the patient condition information when comparing the body temperature with the reference temperature, setting the reference temperature based on the patient condition information when comparing the body temperature with the reference temperature, or increasing or decreasing the comparison result based on the patient condition information; and
determining the risk related to the health status of the patient based on the corrected body temperature.
